(19) United States
(12) Patent Application Publication
(10) Pub. No.: US 2011/0206617 A1
(43) Pub. Date: Aug. 25, 2011

(54) MODIFIED POLYSACCHARIDES FOR DRUG AND CONTRAST AGENT DELIVERY

(76) Inventors: Krishnendu Roy, Austin, TX (US); Bilal Ghosn, Baton Rouge, LA (US); Rebecca Richards-Kortum, Houston, TX (US); Anne van de Ven-Moloney, Houston, TX (US)

(21) Appl. No.: 13/032,280

(22) Filed: Feb. 22, 2011

Related U.S. Application Data
(60) Provisional application No. 61/338,634, filed on Feb. 22, 2010.

(51) Int. Cl. A61K 49/00; A61K 47/36; A61K 39/395; C08B 37/08; A61P 43/00; A61P 35/00; B82Y 5/00

(52) U.S. Cl. 424/9.6; 514/777; 424/130.1; 424/9.1; 536/20; 977/773

(57) ABSTRACT

According to one embodiment, the disclosure provides a delivery formulation including a chitosan in which the primary amines are partially modified with 4-imidazole acetic acid monohydrochloride and a therapeutic or imaging material. According to another embodiment, the disclosure provides a method for delivering a material to an epithelial or mucosal tissue comprising increasing the permeability of an epithelial or mucosal tissue by applying to said tissue a delivery formulation including a chitosan in which the primary amines are partially modified with 4-imidazole acetic acid monochlorohydride and a therapeutic or imaging material.

### CROSS-REFERENCE TO RELATED APPLICATIONS

The application claims the benefit of U.S. Provisional Application Ser. No. 61/338,634, filed Feb. 22, 2010, the entire disclosure of which is hereby incorporated by reference.

### STATEMENT OF GOVERNMENT INTEREST

The present invention was made with support under Grant Number CA103830 awarded by National Institutes of Health. The U.S. government has certain rights in the invention.

### BACKGROUND

Early detection is critical for reducing cancer morbidity and mortality. Optical imaging and spectroscopy can be used to interrogate biochemical and architectural changes associated with early epithelial neoplasia to identify suspicious lesions. Molecular-specific optical contrast agents, capable of selectively labeling biomarkers up-regulated during neoplastic progression, have received much interest for their potential to improve early detection. When interrogated optically, these contrast agents can provide dynamic, real-time information about important molecular hallmarks of neoplasia. Studies of small molecule optical probes with tissue-permeant properties, such as fluorescent sugar derivatives and nucleic acid dyes, have highlighted the promise of using optical imaging to distinguish molecular changes in small populations of cells.

In recent years tremendous progress has been made in developing sophisticated, molecularly-targeted contrast agents, including antibodies, quantum dots and various metal nanosensors (e.g. gold nanoparticles, nanorods, nanoshells etc.). It has been also demonstrated that targeted gold nanoparticles can provide high contrast between normal and pre-cancerous epithelial tissues. However, their clinical applicability, as a topically applied diagnostic agent for early detection of cancer is severely limited, due to our inability to efficiently deliver these agents across mucosal surfaces. Morphological changes associated with precancer generally begin in the basal layers of epithelium, thus early detection strategies require contrast agents to be delivered through several hundred microns of epithelial tissue. It is important that these agents be delivered efficiently and uniformly throughout the region of interest ensuring they reach and bind to their targets. In addition, unbound agents should also be easily “washed out” to reduce non-specific signaling and false positive detection. The penetration of molecules through mucosal tissue depends in part on their size and generally decreases exponentially with increasing hydrodynamic volume. Chemical modification and encapsulation strategies to improve tissue penetration have only proven useful for molecules up to 6 kDa in size.

Permeation enhancers (also called penetration or absorption enhancers) have been investigated to facilitate the delivery of larger molecules. These are topically applied substances that disrupt epithelial tissue by various mechanisms to increase paracellular (between cell) and/or transcellular (across cell) transport. Numerous permeation enhancers have demonstrated efficacy in skin, allowing trans-dermal penetration of drugs, macromolecules, nano- and microparticles as large as 1 to 5 μm. However, studies utilizing similar formulations in the mucosal epithelium have only enhanced the penetration of small molecules, including insulin (6 kDa), calcitonin (3.5 kDa), and dextrans up to 70 kDa in weight. The surfactant Triton-X100 has recently demonstrated reversible mucosal permeation enhancement for efficient delivery of molecules up 150 kDa in size. Nevertheless, the slow rate of tissue recovery associated with Triton-X100 limits the clinical feasibility of this approach as morphological and histochemical analysis of tissues following treatment with the permeabilizing agent is not highly practical.

Chitosan and its analogs have been studied extensively for in vivo delivery of DNA and siRNA via mucosal delivery routes. Chitosan is a biocompatible, biodegradable, cationic polysaccharide comprised of repeating units of glucosamine and N-acetyl glucosamine. It has been shown to reversibly disrupt epithelial tight junctions to allow the paracellular transport of small molecules. Topical application of chitosan has been shown to enhance drug uptake in nasal, buccal, and intestinal epithelia and its bioactivity is mediated by its cationic properties. The ability of chitosan to enhance the permeation of tissue is highly influenced by the pH of the environment. Chitosan is insoluble at neutral pH but is soluble and positively charged at acidic pH. This leads to a major drawback of chitosan for clinical use due to its limited solubility at physiological pH and above, which also leads to loss of charge. To improve these characteristics and enhance its permeation properties, a molecular analog was developed in which the primary amines are partially modified with 4-imidazole acetic acid monohydrochloride (chitosan-IAA) thereby introducing secondary and tertiary amines to the polymer structure. Chitosan-IAA can enhance pDNA expression and siRNA-mediated knockdown in cells when compared to non-modified chitosan. Chitosan-IAA and these nucleic-acid uses are described in PCT/US2008/075799, incorporated by reference herein.

### SUMMARY

The present disclosure generally relates to methods for the topical delivery of biologics and contrast agents. More particularly, the present disclosure relates to methods for the topical delivery of biologics and contrast agents utilizing an imidazole-functionalized conjugate of chitosan.

In one embodiment, present disclosure provides a method of using chitosan with imidazole acetic acid monohydrochloride conjugated to the primary amines to deliver drugs, imaging agents, or other materials to mucosal or epithelial cells.

In another embodiment, the present disclosure provides a delivery formulation including a chitosan in which the primary amines are partially modified with 4-imidazole acetic acid monohydrochloride and a non-nucleic acid therapeutic or imaging material.

In another embodiment, the present disclosure provides a method for delivering a material to an epithelial or mucosal tissue comprising increasing the permeability of an epithelial or mucosal tissue by applying to said tissue a delivery formulation including a chitosan in which the primary amines are partially modified with 4-imidazole acetic acid monochlorohydride and a non-nucleic acid therapeutic or imaging material.

The features and advantages of the present invention will be apparent to those skilled in the art. While numerous changes may be made by those skilled in the art, such changes are within the spirit of the invention.

### DRAWINGS

Some specific example embodiments of the disclosure may be understood by referring, in part, to the following description and the accompanying drawings.

FIG. 1 is a chemical schematic for the synthesis of imidazole-modified chitosan (chitosan-IAA).

FIG. 2 shows the stromal accumulation of fluorescent macromolecules following topical permeation treatment. Freshly excised bladder tissue was treated for 15 minutes with 0.01% w/v chitosan-IAA, chitosan, or media and then probed with a 1:1:1 mixture of 3 kDa rhodamine-dextran, 40 kDa fluorescein-dextran, and Alexa647-IgG. The yellow/white lines indicate the boundary between the epithelium and stroma. In reflectance images, the epithelium was distinguished from the stroma by its darker appearance. Following permeation treatment, the fluorescent macromolecules accumulated in the stroma. Stromal accumulation was brighter following treatment with imidazole-modified chitosan than with non-modified chitosan. In media-treated controls superficial epithelium. The scale bar represents 100 μm. The epithelium (E) and stroma (S) are labeled in the first image.

FIG. 3 stromal accumulation of gold nanoparticles following topical permeation treatment. Freshly excised bladder tissue was treated for 15 minutes with 0.01% w/v chitosan-IAA, chitosan, or media and then fluorescein-PEG-gold spheres of 44 and 33 nm diameter were applied topically. Trans-epithelial delivery of both sizes of nanoparticles was observed by monitoring for the localization of nanoparticle-associated fluorescence. The stromal reflectance was visibly enhanced in samples showing accumulation of nanoparticles. No significant change in stromal fluorescence or reflectance was observed in media-treated controls. The scale bar represents 100 μm.

FIG. 4 shows a representative image, taken at a depth of 10 μm, of optical sectioning of bladder epithelium and stroma following the topical application of 3 kDa rhodamine-dextran. The mucosal surface was pre-treated with chitosan-IAA30 for 15 minutes at 37° C. Confocal fluorescence images were acquired parallel to the tissue surface in 2 μm steps with constant laser power and gain. Rhodamine-dextran permeation through the epithelium of the tissue appears to follows a paracellular route, appearing as bright rings around cells and minimal labeling within the cells. As the imaging progresses deeper the stroma appears, clearly defined by a selective accumulation of contrast agents. The scale bar represents 100 μm.

FIG. 5 shows a representative image, taken at a depth of 10 μm, of optical sectioning of bladder epithelium and stroma following the topical application of 44 nm fluorescein-PEG-gold. The mucosal surface was pre-treated with chitosan-IAA30 for 15 minutes at 37° C. Confocal fluorescence images were acquired parallel to the tissue surface in 2 μm steps with constant laser power and gain. Nanoparticle transport in the epithelium appears to follow both paracellular and transcellular routes, appearing as bright rings around cells and diffuse labeling within cells. Individual nuclei appear as a black spot within each cell. Deeper imaging within the tissue samples demonstrates clear nanoparticle uptake throughout the stromal regions. The scale bar represents 100 μm.

FIG. 6 shows fluorescence intensity as a function of time and temperature. Tissues pre-treated with chitosan-IAA were washed and allowed to recover at 37° C. (FIG. 6A) or 4° C. (FIG. 6B). The recovery of barrier function was probed at regular time intervals using fluorescent macromolecules and nanoparticles. Tissues held at 37° C. rapidly recovered barrier function while tissues held at 4° C. displayed continued permeability as evidenced by stromal fluorescence. The ability of the recovering epithelium to block contrast agents was size dependent, with larger molecules excluded more rapidly than smaller molecules.

FIG. 7 shows representative confocal images of antiEGFR-647 labeling in fresh human oral biopsies co-treated with chitosan-IAA, collected at the same gain. Biopsies were topically treated with antiEGFR-647 diluted in 0.01% w/v chitosan-IAA for 1 hour at 37° C., washed 3 times, sliced transversely, and imaged. In normal tissues (top), EGFR labeling is limited to basal layer of epithelial cells, appearing as a faint region of labeling at the base of the epithelium. Non-specific labeling is observed at the apical surface of keratinized tissues such as the retromolar trigone tissue shown below. EGFR labeling in squamous cell carcinoma (bottom) is characterized by bright, ring-like extracellular labeling extending downwards from the tissue surface. The scale bar represents 100 μm.

FIG. 8 shows the measured mean fluorescence intensity of paired oral biopsies collected from patients with cancer of the buccal (1), retromolar trigon (2), maxilla (3, 4) and tongue (5) regions. Biopsies were collected from clinically abnormal and contralateral normal regions. The biopsy pairs were topically labeled for EGFR in the presence of chitosan-IAA and imaged transversely as shown in FIG. 7. Statistically significant differences (P<0.01) were observed in mean fluorescence between biopsy pairs.

FIG. 9 is a schematic of carbodiimide reaction for conjugation of IAA to chitosan.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to methods for the topical delivery of biologics and contrast agents. More particularly, the present disclosure relates to methods for the topical delivery of biologics and contrast agents utilizing an imidazole-functionalized conjugate of chitosan.

In some embodiments, the current disclosure relates to the use of chitosan in which the primary amines are partially modified with 4-imidazole acetic acid monohydrochloride (chitosan-IAA) thereby introducing secondary and tertiary amines to the polymer structure. The secondary and tertiary amines may increase the buffering ability and solubility of the chitosan. The chitosan-IAA may be used to deliver materials to mucosal or epithelial cells in vivo or in situ. Chitosan-IAA is described in further detail in PCT/US2008/075799, incorporated by reference herein.

According to one particular embodiment, chitosan-IAA may reversibly enhance mucosal permeability. More specifically, the chitosan-IAA may also cause minimal to no tissue damage. Tissue treated with chitosan-IAA may show a rapid tissue recovery. The speed of this tissue recovery, which relates to return to normal tissue permeability may be balanced against the material delivery needs, which may rely on increase in tissue permeability by the chitosan-IAA. In particular, the concentration of the chitosan-IAA, material delivered, formulation, duration of exposure of tissue to chitosan-IAA, and active removal or suppression of natural removal processes may all be varied. Chitosan-IAA may increase uptake of the delivered material by epithelial or mucosal cells by transcellular or paracellular routes. The route of uptake may be determined by the size of the material.

In particular embodiments, the chitosan-IAA and material may be delivered to mucosal membranes and surrounding tissues. For example, the chitosan-IAA and material may be delivered to cheek or other oral tissue or to bladder tissue. In some embodiments the chitosan-IAA and material may be formulated for topical application. In other embodiments, it may be formulated for internal use.

For in situ use, the chitosan-IAA material may be formulated in a gel or liquid for administration to a biopsied tissue. The material may then be detected in the biopsied tissue.

The material delivered with the chitosan-IAA may be a non-nucleic acid material. For example, it may be a macromolecule, drug, contrast agent or a nanoparticle. Small molecules and nanoparticles may be up to 44 nm in size. Macromolecules may be between 4 kDa and 150 kDa. Macromolecules may include proteins, such as antibodies and peptides. Nanoparticles may include metal nanoparticles, such as gold nanoparticles, which may include surface-modified gold nanoparticles. Drugs may be a macromolecule or a small molecule. Contrast agents may be optical contrast agents and may also be a macromolecule or a small molecule. Materials may be neutral in charge. However, charge neutrality may be less important in materials that pre-treated with chitosan-IAA. The chitosan-IAA may be formulated into combinations with multiple materials, such as drug-nanoparticle combinations. Chitosan-IAA may have targeting agents or adjuvants attached or co-delivered as well.

According to one embodiment, the chitosan-IAA may itself be formulated as a nanoparticle either alone or in combination with the material.

The chitosan-IAA may be attached to the material, for example through covalent or ionic bonding. In some embodiments, chitosan-IAA may be presented with some material bound and some not. This may allow selective control of the material delivery route, which may be useful for distinguishing between true intracellular labeling and endocytic uptake.

In imaging applications, an optical imaging material may be delivered. This optical imaging material may have or produce a certain color detectable by microscopy, such as confocal microscopy.

According to a specific embodiment, chitosan-IAA may facilitate rapid trans-epithelial delivery of both macromolecules and nanoparticles in mucosal tissue. In particular, the stromal accumulation of these agents may be higher following chitosan-IAA treatment. Delivery may be improved by chitosan-IAA pre-treatment and co-treatment. Complexation of the delivered material with chitosan-IAA may not be necessary for enhanced penetration.

Without limiting the disclosure to one mode of action, the mechanism by which chitosan and its analogs increase tissue permeability may be mediated by positive charges on the chitosan. It may include interactions with tight junction proteins, the redistribution of F-actin, and a destabilization of the cell membrane. Tissue permeability increase may be an active, energy-dependent process because no trans-epithelial contrast agent delivery was observed following permeation treatment at 25° C. or 4° C. in the examples below. Similarly, the recovery of barrier function may be an active process, requiring incubation at 37° C. after washing of the tissue. Permeation of bladder mucosa is rapid, with a 15-minute treatment sufficient to facilitate contrast agent transport through several layers of epithelial cells.

According to another embodiment, processes using chitosan-IAA may be regulated using temperature. The gain and loss of tissue permeability in the presence of chitosan-IAA can be halted simply by placing the tissue on ice. Tissues chilled at particular time-points during the recovery process may preferentially exclude larger contrast agents more rapidly than smaller contrast agents. The ability of the treated tissues to block trans-epithelial agent delivery increases progressively.

The ability of chitosan to enhance the permeation of tissue may also be influenced by the pH of the environment. Chitosan is insoluble at neutral pH but is soluble and positively charged at acidic pH. It is believed that the increased solubility and buffering capacity of chitosan modified with 4-imidazole acetic acid monohydrochloride improves trans-epithelial contrast agent delivery.

According to another embodiment, processes using chitosan-IAA may have increased recovery of epithelial barrier function if the epithelial tissue is washed after chitosan-IAA treatment. In vivo the chitosan-IAA may naturally be removed or diluted rapidly enough to allow restoration of epithelial barrier function. In some in vivo applications, reapplication of the chitosan-IAA or measures to reduce its natural removal may be useful. Chitosan-IAA removal may be sufficiently rapid to minimize the entry of toxins or allergens through the permeabilized epithelium. Efficient delivery has been observed following both chitosan-IAA pre-treatment and co-treatment, suggesting that complexation with chitosan-IAA is not necessary for enhanced penetration.

In one embodiment, chitosan-IAA may be used to deliver tissue-impermeant optical contrast agents through the epithelium of freshly resected mucosal tissues. In certain embodiments, chitosan-IAA may be used to reversibly enhance mucosal permeability in a rapid, reproducible manner sufficient to facilitate the trans-epithelial delivery of optical contrast agents up to 44 nm in diameter. In certain embodiments, permeation enhancement may occur though an active process, resulting in the delivery of contrast agents via a paracellular or a combined paracellular/transcellular route depending on size.

The rapid reversibility of chitosan-IAA mediated permeation supports the clinical potential of this approach. There are concerns that topical permeation enhancers, which by nature interfere with the barrier function of the epithelium, could potentially permit the entry of toxins and/or allergens. Based on results, it is expected that the penetration of these agents to be blocked rapidly. Compared to the topical permeation enhancer Triton-X100, tissue recovery following chitosan-IAA treatment is much more rapid.

There are several factors which determine the route of contrast agent penetration. Chitosan solutions have been shown to increase both the paracellular and transcellular permeability of CaCo-2 cell monolayers in a reversible, dose-dependent manner. Contrast agents up to 150 kDa in size appeared to follow primarily a paracellular route, while nanoparticles followed both paracellular and transcellular routes. Without wishing to be limited to theory, it is believed that the observed paracellular transport resulted from an opening of the tight junctions and the observed transcellular transport was a consequence of cell membrane destabilization. Both of these mechanisms have been described in CaCo-2 monolayers following chitosan treatment. Without wishing to be limited to theory, it is believed that the shift towards a transcellular route may be determined by probe size. The formulation of chitosan into nanoparticle-drug complexes has been demonstrated to enhance the internalization of chitosan in cells and tissues, presenting a second potential approach for driving transcellular delivery.

Controlled, uniform delivery of molecular-specific contrast agents across the full-thickness of the epithelium remains an important goal for design of early detection strategies. One-time topical application of 0.1% w/v chitosan-IAA facilitates the permeation enhancement in a variety of tissues, including the transitional epithelium of the bladder, squamous epithelium of the oral cavity, and squamous cell carcinoma. Delivery of fluorescent EGFR-specific antibodies can be observed across the full thickness of the epithelium (150-400 μm) in all normal oral biopsies. Differential labeling may be observed between normal and cancer specimens, suggesting that the specificity of the antibody and the structure of the target are conserved in the presence of chitosan-IAA. This is in agreement with studies of EGFR labeling in live cell monolayers in the presence and absence of 0.1% w/v chitosan-IAA, where minimal loss in labeling of antibody labeling was seen at the equivalent of a 0.1% w/v treatment. The observed differential contrast ratio varies from patient-to-patient, but EGFR labeling in cancer samples is on average 11 times greater than in normal samples.

To facilitate a better understanding of the present invention, the following examples of certain aspects of some embodiments are given. In no way should the following examples be read to limit, or define, the entire scope of the invention.

### EXAMPLES

### Example 1

### Chemical Modification of Chitosan

Chitosan was chemically modified to improve its solubility and tissue permeation capacity. The conjugation of 4-imidazole acetic acid monohydrochloride (IAA) to the primary amines of chitosan for production of modified chitosan-IAA was performed. Briefly, 86% deacetylated chitosan of 130 kDa molecular weight (PCL 113, Novamatrix, Norway) and IAA (Acros Organics, Morris Plains, N.J.) were dissolved in 0.1 M 2-(N-morpholino)ethanesulfonic acid (pH 6.5, Sigma-Aldrich, St Louis, Mo.) to a concentration of 1.0% and 2.0% w/v respectively. A theoretical modification of 50% was targeted for the reaction. IAA and chitosan were combined at a volumetric ratio of 1:5 while on ice. 20 M excess (in relation to IAA) of 1-ethyl-3-(3-dimethylaminopropyl carbodiimide hydrochloride (Peirce Biotechnology, Rockford, Ill.) was added and immediately vortexed to promote addition of IAA to the chitosan backbone. Reactions were allowed to continue overnight with end-over-end mixing. Solutions were dialyzed for 2 days against 5 mM hydrochloric acid (Acros Organics) thrice and then deionized water thrice using Snakeskin pleated dialysis tubing (10,000 MW cut-off; Pierce Biotechnology, Inc., Rockford, Ill.). Following dialysis, the samples were lyophilized for 24 hours. Degree of substitution was determined with via ¹H NMR. Chitosan and chitosan-IAA were resuspended at a stock concentration of 1% w/v in 0.2M sodium acetate (pH 4.5, Sigma Aldrich) immediately prior to dilution for tissue application.

### Example 2

### Synthesis and Validation of Gold Nanoparticles

Gold nanoparticles were synthesized as untargeted tissue permeability probes. Gold spheres of 9.5±0.35 nm and 20.7±0.26 nm hydrodynamic diameter were synthesized by a citrate reduction of tetrachloroauric (III) acid (HAuCl₄, Sigma Aldrich, St. Louis, Mo.) under reflux. Nanoparticle size was confirmed by dynamic light scattering (DLS) with a ZetaPlus system (Brookhaven Instruments Corporation, Holtsville, N.Y.). Each set of gold nanoparticles were coated with a fluorescent 5 kDa polyethylene glycol (PEG) to reduce their aggregation in media. Briefly, 0.1% w/v fluorescein-PEG-amine (Laysan Bio, Arab, Ala.) was reacted with 2 M excess of 2-iminothiolane (Pierce Biotechnology) for 2 hours. Upon completion, the solution was filtered with Amicon Ultra centrifugation filters (3,000 MWCO; Millipore, Billerica, Mass.) at 3,220×g. Gold nanoparticles, suspended at a concentration of 7×10¹⁰ particles/ml (determined by spectral absorbance analysis), were allowed to react with 10⁻⁴ M fluorescein-PEG-thiol at a volumetric ratio of 5:1 for 45 minutes on a shaker for each set of nanoparticles. PEG (Sigma Aldrich, St. Louis, Mo.) was subsequently added to a final concentration of 2% w/v. The particles were centrifuged at 1500×g for 30 minutes and resuspended in deionized water. The successful coating of the nanoparticles was confirmed by monitoring for ˜494 nm absorbance peak using a Synergy HT UV-Vis spectrophotometer (Biotek, Winooski, Vt.). The final hydrodynamic radius of the gold nanoparticles was measured to be 32.9±1.27 nm and 43.9±0.51 nm using DLS demonstrating similar size increase following PEGylation as was previously described.

### Example 3

### Synthesis and Validation of Fluorescent Macromolecules

Fluorescent macromolecules were prepared as targeted and untargeted tissue permeability probes. 3 kDa rhodamine-dextran, 40 kDa fluorescein-dextran, and 150 kDa AlexaFluor 647-IgG were purchased from Invitrogen (Carlsbad, Calif.) for use as untargeted probes. For targeted labeling studies, mouse anti-human antibodies specific for epidermal growth factor receptor (EGFR; clone 108; custom synthesized by the Baylor College of Medicine, Houston, Tex.) were reacted with AlexaFluor 647 carboxylic acid succinimidyl esters using commercially available labeling kits (Invitrogen). The purified conjugates were suspended in PBS at a concentration of 1.0 mg/ml. Dye-labeled isotype controls were synthesized at the same concentrations. Prior to tissue labeling, the bioactivity and specificity of the conjugates was confirmed using live EGFR-positive 1483 cells and EGFR-negative MDA-MB-4355 cells as described in, both in the presence and absence of chitosan and chitosan-IAA.

### Example 4

### Topical Permeation of Fresh Bladder Tissue

Guinea pig bladder mucosa was used as a model tissue to evaluate tissue permeability in the presence and absence of chitosan-based permeation enhancers because of the folding nature of the tissue. The guinea pig bladder allows for effective imaging of both epithelial and stromal layers of the tissue at once. All animals were cared for in accordance with institutional guidelines. The protocols were reviewed and approved by the IACUC at Rice University. This model has been previously validated for the study of topical permeation enhancement. Whole bladders were excised from 3-4 week old female Hartley guinea pigs (Charles River Laboratories, Wilmington, Mass.) directly following animal sacrifice. The bladders were sectioned into 8-10 pieces of uniform surface area (3×3 mm) using a scalpel and washed once in PBS and then immediately used for permeation studies. Two topical permeation enhancers, chitosan and chitosan-IAA, diluted to 0.1% w/v in DMEM/F12 medium (Invitrogen), were evaluated for their ability to increase tissue permeability. Media alone was used as a negative control. 100 μL of permeation enhancing solution was topically applied to the apical surface of each tissue biopsy for 15 minutes.

### Example 5

### Assessment of Trans-Epithelial Macromolecule Delivery

To determine whether chitosan-treated tissues are selectively permeable to macromolecules of specific sizes, fluorescent macromolecules ranging from 3 kDa to 150 kDa in size were topically applied to the tissue surfaces of bladder biopsies. Tissue penetration was then monitored optically. The apical surface of the epithelium was topically treated for 15 minutes with pre-warmed (37° C.) 0.1% w/v chitosan or chitosan-IAA, washed once in cold media, and covered with a 1:1:1 mixture of 3 kDa rhodamine-dextran, 40 kDa fluorescein-dextran, and 150 k Da AlexaFluor 647-IgG, each diluted to a concentration of 1 μM in cold media. Tissues were immersed with this solution for 15 minutes at 4° C. and then imaged using confocal microscopy at three different excitation wavelengths (described below). Images were collected in 2-5 μm steps from the surface into the tissue. Following imaging, the tissues were washed 3 times in cold media (15 minutes total) and re-imaged to assess the removal of unbound macromolecules. Experiments were repeated with chitosan-IAA treatment at 25° C. or 4° C. to determine the influence of temperature on tissue permeation. Each labeling condition was evaluated in 6 independent experiments.

### Example 6

### Assessment of Trans-Epithelial Nanoparticle Delivery

To determine whether chitosan-treated tissues permit the trans-epithelial delivery of nanoparticles, nanoparticles of different sizes were topically applied to the surface of permeabilized bladder biopsies. Nanoparticle delivery was then monitored optically. Briefly, the apical surface of the epithelium was topically treated for 15 minutes with pre-warmed (37° C.) 0.1% w/v chitosan or chitosan-IAA diluted in DMEM/F12 medium, washed once in cold media, and covered with fluorescein-PEG-gold spheres of 33 or 44 nm diameter, each diluted to a concentration of 1×10⁶ nanoparticles/ml in cold media. Tissues were immersed with this solution for 15 minutes at 4° C. and then imaged using confocal microscopy. Images were collected in 2-5 μm steps from the surface into the tissue. Following imaging, the tissues were washed 3 times in cold media (15 minutes total) and re-imaged to assess the removal of unbound nanoparticles. Each labeling condition was evaluated in 6 independent experiments.

### Example 7

### Time-Course Analysis of Tissue Recovery

To monitor the recovery of bladder epithelial barrier function following the removal of chitosan-IAA, fresh bladder biopsies were treated with 0.1% chitosan-IAA or media for 15 minutes at 37° C., washed, and probed with non-targeted contrast agents at regular time-intervals during tissue recovery. Briefly, chitosan-IAA and media-treated samples were washed 3 times with warm or cold media and then allowed to recover in media at 37° C. or 4° C. At 0, 15, 30, 60, 90, or 120 minutes after chitosan-IAA removal, the samples were placed on ice and topically labeled with a 1:1:1 mixture of untargeted fluorescent macromolecules (3 kDA dextran, 40 kDa dextran, and IgG antibody) or 44 nm gold nanoparticles. Samples were allowed to incubate with the macromolecules or nanoparticles for 15 minutes prior to imaging via confocal microscopy. Images were collected 40 μm below the tissue surface to allow for optical sectioning of both the epithelium and underlying stroma. Tissue samples were assessed in duplicate using a minimum of 3 independent experiments.

### Example 8

### Molecular-Specific Labeling of Human Oral Biopsies

To demonstrate the feasibility of delivering molecular-specific optical contrast agents targeted against biomarkers of neoplasia, human oral biopsies were co-treated with fluorescent EGFR antibodies and chitosan-IAA. Briefly, paired clinically normal and abnormal biopsies of the oral mucosa were obtained from 5 consenting patients at the University of Texas M. D. Anderson Cancer Center (MDACC). Patients gave written informed consent, and the clinical protocols were approved by the Institutional Review Boards at MDACC and Rice University. Biopsies were immediately placed and remained in chilled media until they arrived at Rice University. The biopsies were embedded vertically into 3% w/v ultrapure agarose (Invitrogen) to prevent the influx of permeation enhancers and contrast agents at the tissue margins as previously described. The apical surface of the epithelium was left free of agarose to facilitate topical labeling. Based on the increased number of epithelial layers in human oral resections, tissues were topically treated with 1 μM antiEGFR-647 diluted in 0.1% w/v chitosan-IAA for 60 minutes at 37° C., washed 3 times with cold media, and then sliced transversely into 200 μm thick slices using a Krumdieck tissue slicer. Tissue slices were counterstained with 0.01% proflavine, a nucleic acid dye, and immediately imaged using confocal microscopy.

### Example 9

### Confocal Image Acquisition

All images were obtained using a Carl Zeiss LSM 510 confocal microscope (Thornwood, N.Y.) equipped with 488 nm (30 mW), 543 nm (1 mW), and 633 nm (5 mW) lasers. Images were collected using PMT detectors and Zeiss LSM 5 image examiner software. Samples were sequentially excited with each laser line with power settings held constant for each laser. Fluorescence emission was collected using 500-530 nm (40 kDa fluorescein-dextran), 565-615 nm (3 kDa rhodamine-dextran), and 650-710 nm (150 kDa Alexa647-IgG) band-pass filters. Tissue reflectance at 633 nm was collected using a 635 nm dichroic beam splitter. Images were acquired at 0.5 frames per second using a 20× objective with a pinhole of 2.56 Airy units. For the untargeted macromolecule and nanoparticle permeation studies, the gain was held constant with excitation at 488, 543, and 633 nm. At the gains utilized, the fluorescence of solutions outside the tissue was oversaturated, but the fluorescence of solutions within the tissue was not. In the EGFR targeting studies, the gain was held constant for antiEGFR-647 imaging (633 nm excitation) and proflavine imaging (488 nm excitation).

### Example 10

### Image Analysis

To quantify the fluorescence intensity of the stroma as a function of recovery time and temperature, representative confocal fluorescence images were analyzed using Image J v1.38 (NIH, Bethesda, Md.). The mean fluorescence intensity per unit area was determined by selecting regions of interest (ROI) within each image and then dividing the measured fluorescence intensity by the area of the ROI. The ROI margins were drawn just inside the stroma border as determined from reflectance overlay images. The entire stroma was included for each image. When images contained more than one region of discontinuous stroma, the mean intensity for the regions was averaged and treated as a single value. Background noise was determined at each time-point from samples pre-treated with media in place of chitosan-IAA. The normalized mean fluorescence intensity of the stroma was calculated by subtracting the measured background and normalizing for the fluorescence intensity of the stroma at time of chitosan-IAA removal. The normalized mean fluorescence intensity of the stroma was assessed for each macromolecule using 5 representative images from 3 independent experiments (15 images total per macromolecule per time-point and temperature).

The mean reflectance intensity per unit area of the stroma in nanoparticle labeling studies was quantified in the same manner as described for the fluorescence studies, using 5 representative images from 5 independent experiments (25 images total per labeling condition).

To quantify the fluorescence intensity of EGFR labeling in transverse images in human oral biopsies, representative confocal fluorescence images were analyzed. In normal samples, ROIs were bounded by the apical and basal surfaces of the epithelium, thereby including any labeling throughout the epithelium. In neoplastic samples, ROIs were drawn to include all squamous cells from the surface of the tissue to the lower margin of EGFR labeling. Regions of fibroblasts, as identified morphologically by proflavine counterstaining, were excluded from the analyses. The mean fluorescence intensity per unit area was assessed using 15 images per biopsy, consisting of 3 representative images from 5 separate tissue slices. No background corrections were made because the measured background was <1% of the measured mean EGFR labeling intensity for all images evaluated. Differences in mean labeling intensity between normal and neoplastic samples were assessed on a patient-to-patient basis using a two-tailed, unpaired Students t-test, with p-values of ≦0.01 being considered statistically significant. The relative contrast ratio was determined by dividing the mean fluorescence intensity of each cancer biopsy by that of the contralateral normal.

Results

Synthesis and Characterization of Imidazole-Modified Chitosan

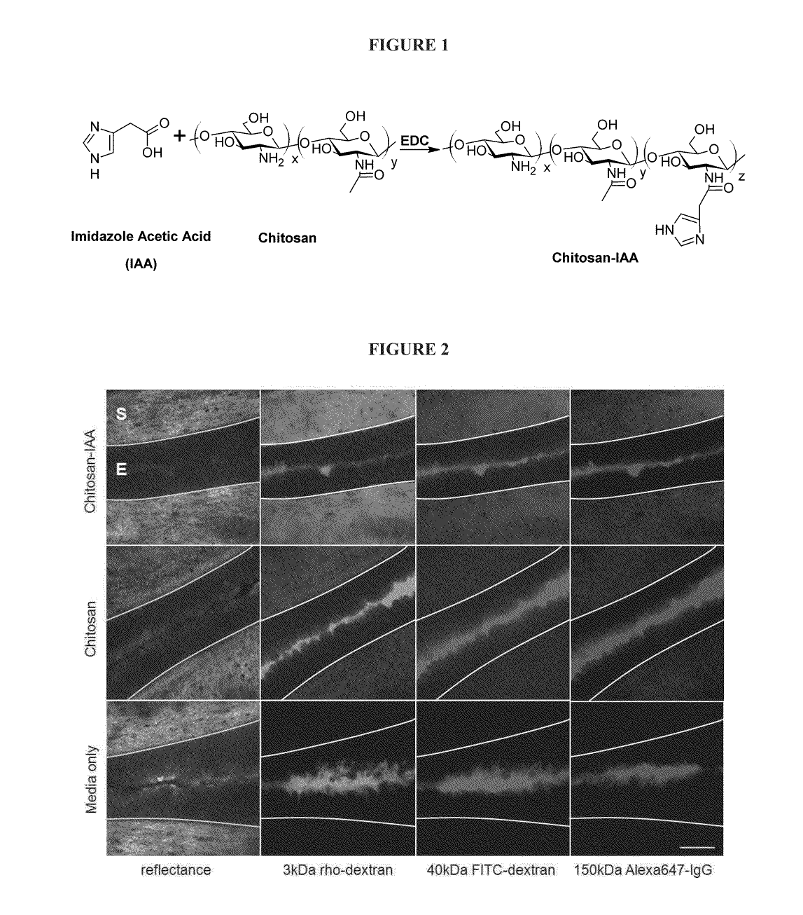

A schematic for the synthesis of chitosan-IAA is shown in FIG. 1. We successfully introduced imidazole acetic acid to the primary amines of chitosan via a carbodiimide-mediated reaction. Following synthesis, ¹H NMR characterization of the purified polymer provided on average a degree of substitution of about 3.0% of the primary amines modified for each chitosan molecule. Chitosan-IAA polysaccharide prepared through this type of reaction has been previously shown to provide enhanced polymer solubility and buffering capacity.

Trans-Epithelial Macromolecule Delivery Through Fresh Bladder Mucosa

FIG. 2 shows representative confocal fluorescence images of bladder tissue treated topically with 0.1% w/v chitosan-IAA, chitosan, or media for 15 minutes 37° C. followed by topical application of a 1:1:1 mixture 3 kDa rhodamine-dextran, 40 kDa fluorescein-dextran, and 150 kDa Alexa647-IgG. The tissue reflectance images are shown on the left and the corresponding fluorescence images are shown to the right. The yellow/white lines indicate the boundary between the stroma and the epithelium. Due to the three-dimensional folding of the resected bladder, it was possible to image the epithelium in cross-section using confocal microscopy at a depth of 40 μm. In the reflectance images, the epithelium was distinguished from the stroma by its darker appearance. Both chitosan and chitosan-IAA were found to facilitate the trans-epithelial delivery of macromolecules. Macromolecules of all three sizes accumulated in the stroma of permeation-enhanced tissues, causing the stroma to appear brighter than the epithelium in the fluorescence images. Chitosan-IAA treatment facilitated more intense stromal accumulation than non-modified chitosan for all sizes of macromolecules evaluated. The macromolecules could be removed with several brief washes in media (data not shown), demonstrating that macromolecule accumulation is reversible. Experiments were independently repeated by labeling tissues with one macromolecule at a time (data not shown) to exclude macromolecule interactions. In media-treated controls, the macromolecule penetration was limited to the superficial epithelium. No significant macromolecule penetration was observed following chitosan-IAA or chitosan treatment at 25° C. or 4° C. (data not shown).

Trans-Epithelial Nanoparticle Delivery Through Fresh Bladder Mucosa

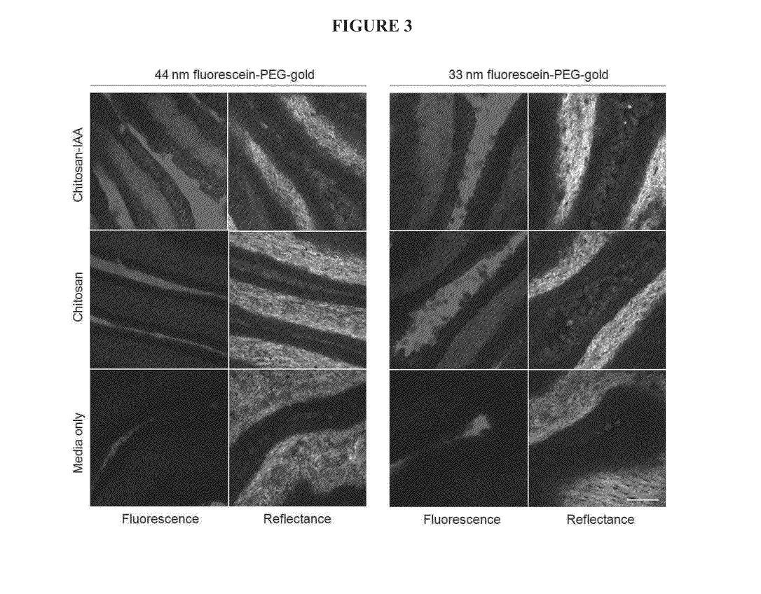

FIG. 3 shows representative confocal fluorescence images of bladder tissue treated topically with 0.1% w/v chitosan-IAA, chitosan, or media for 15 minutes 37° C. and then probed with fluorescein-PEG-gold spheres of 44 or 33 nm diameter. Both chitosan and chitosan-IAA were found to facilitate the trans-epithelial delivery of nanoparticles. Following nanoparticle application, fluorescence was primarily observed in the stroma of permeable tissues. Stromal labeling was generally less uniform than observed with macromolecules. Pre-treatment with chitosan-IAA resulted in more intense stromal accumulation than non-modified chitosan. This difference was more obvious following application of larger nanoparticles. The stromal reflectance was visibly enhanced in samples demonstrating nanoparticle uptake. Tissues exposed to nanoparticles showed a stromal reflectance enhancement of 1.5 to 1.7 (44 nm gold) and 1.7 to 2.1 (33 nm gold) compared to tissues treated with media in place of chitosan following quantification of reflectance at a fixed gain and regions of the stroma. Minimal to no nanoparticle accumulation was observed in media-treated controls.

Route of Contrast Agent Delivery Following Chitosan-IAA Treatment

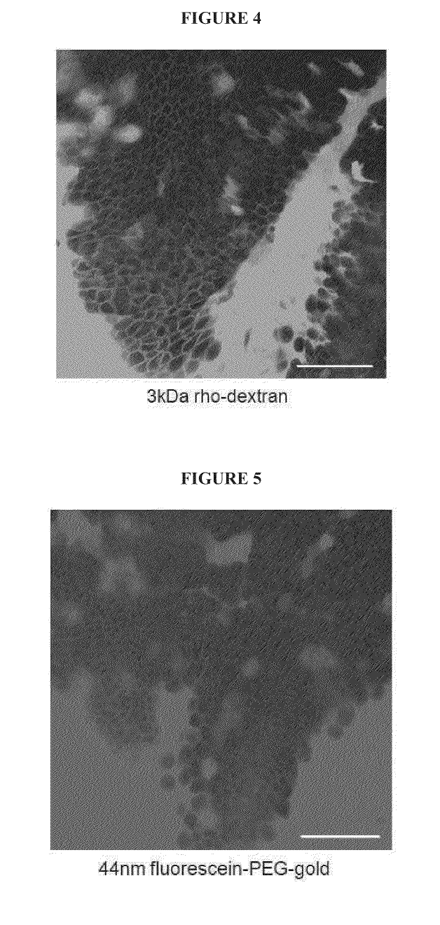

The route of contrast agent delivery through the epithelium was optically interrogated in fresh bladder tissues pre-treated with chitosan-IAA. Confocal fluorescence images were collected in 2 μm steps from the surface of the tissue following the topical application of 3 kDa rhodamine-dextran or 44 nm fluorescein-PEG-gold. Representative videos are available online. The videos start at 2 μm below the surface and advance at rate of 2 μm/second. FIGS. 4 and 5 show the stills for the videos and represent frames collected at 10 μm below the tissue surface. 3 kDa rhodamine-dextran labeling, shown in FIG. 4, was characterized by ring-like fluorescence surrounding each cell in the field of the view. The labeling appeared extracellular, suggesting a paracellular route of dextran delivery. These rings became progressively smaller in diameter with increasing depth in the epithelium, correlating well with known bladder epithelium morphology. In contrast, the delivery of larger particles, shown in FIG. 5 appeared to follow both paracellular and transcellular routes. Fluorescein-PEG-gold delivered in a paracellular manner appeared as ring-like labeling that became progressively smaller with increasing depth. Fluorescence associated with nanoparticles was also observed in the cytoplasm of cells across the full thickness of the epithelium, suggestive of transcellular transport. Individual nuclei appeared as a black spot within each cell, suggesting that the transcellular movement of nanoparticles was limited to the cytoplasmic compartment. In both samples, the transition from epithelium to stroma (first visible at 14-20 μm) was characterized by a relative increase in fluorescence. Dextran and nanoparticle accumulation in the stroma was generally uniform. Few morphological features were defined in stroma, other than blood vessels of horizontal and vertical orientation. These vessels were characterized by low contrast agent accumulation, appearing as dark stripes and circles/ovals respectively.

Epithelial Recovery Following Chitosan-IAA Treatment

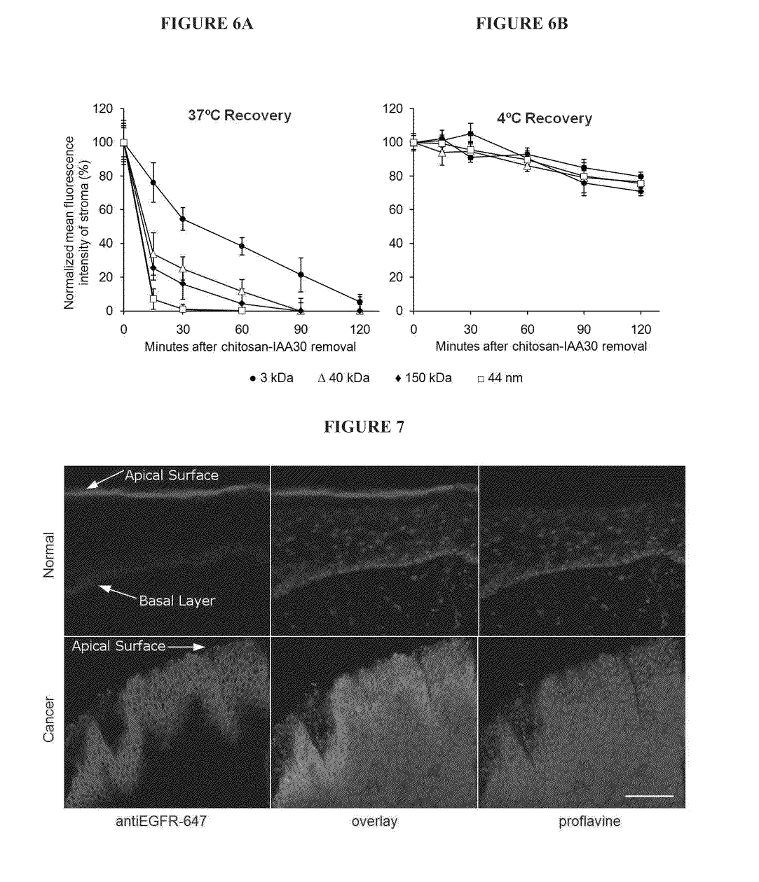

FIG. 6 shows tissue permeability as a function of time and temperature following the removal of chitosan-IAA. Tissue recovery at 37° C. is illustrated on the left and tissue recovery at 4° C. is illustrated on the right. Samples were pre-treated with 0.1% chitosan-IAA for 15 minutes, washed in warm or cold media, and then macromolecules or nanoparticles were applied topically at different time-points. The mean fluorescence intensity of the stroma was measured at each time-point to assess epithelial permeability. At 37° C., the barrier function of the epithelium was found to be recovered quickly, on the order of minutes to hours depending on the size of the permeability probe. The trans-epithelial delivery of 44 nm nanoparticles was reduced by over 90% within 15 minutes of chitosan-IAA removal. By 90 minutes, the epithelium remained permeable (˜22% of initial) only to molecules of 3 kDa in size. With 2 hours of recovery time, the trans-epithelial delivery of 3 kDa molecules was reduced by 95%. Little tissue recovery was observed for samples held at 4° C. Macromolecules and nanoparticles of all sizes continued to accumulate in the stroma at all time-points evaluated. The ability of the macromolecules and nanoparticles to reach the stroma was reduced by only 10% over the course of 2 hours. No significant differences were observed between macromolecules or nanoparticles of different sizes at 4° C. Pre-treated tissue which was not washed and which was kept at 37 C still retained macromolecule permeation for all sizes for up to 2 hours (data not shown).

Delivery of Targeted Contrast Agents for Cancer Detection

To demonstrate the clinical potential of chitosan-IAA to aid in delivery of targeted optical contrast agents for the early detection of cancer biomarkers, paired human oral biopsies were topically labeled with fluorescent EGFR-specific antibodies diluted in 0.1% w/v chitosan-IAA. We have previously demonstrated that antibodies targeted to EGFR lack the ability to penetrate normal oral mucosa and squamous carcinomas. FIG. 7 shows representative transverse sections of normal (top) and cancerous (bottom) gingiva tissue labeled for EGFR and counter-stained with the nucleic acid dye proflavine. Labeling with antiEGFR-647 is displayed in red and proflavine in green. Differential EGFR labeling was observed between normal and cancer samples from all 5 patients evaluated. EGFR labeling in normal epithelium was limited to cells in the basal layer. This labeling was observed in all normal samples evaluated, and was generally less intense and more diffuse than labeling in cancer samples. Non-specific antibody labeling was observed in keratinized tissues, appearing as a bright stripe above the epithelium. EGFR labeling in squamous cell carcinoma tissue was characterized by intense, ring-like labeling extending downwards from the tissue surface. This labeling appeared highly uniform, labeling all squamous cells within the treatment zone. Regions of fibroblasts, which were identified by the presence of small, widely spaced nuclei using proflavine staining, did not show significant EGFR labeling. With one hour of chitosan-IAA treatment, antibody labeling reached a depth of approximately 150 to 250 μm in cancer tissue and ≦400 μm in normal epithelium.

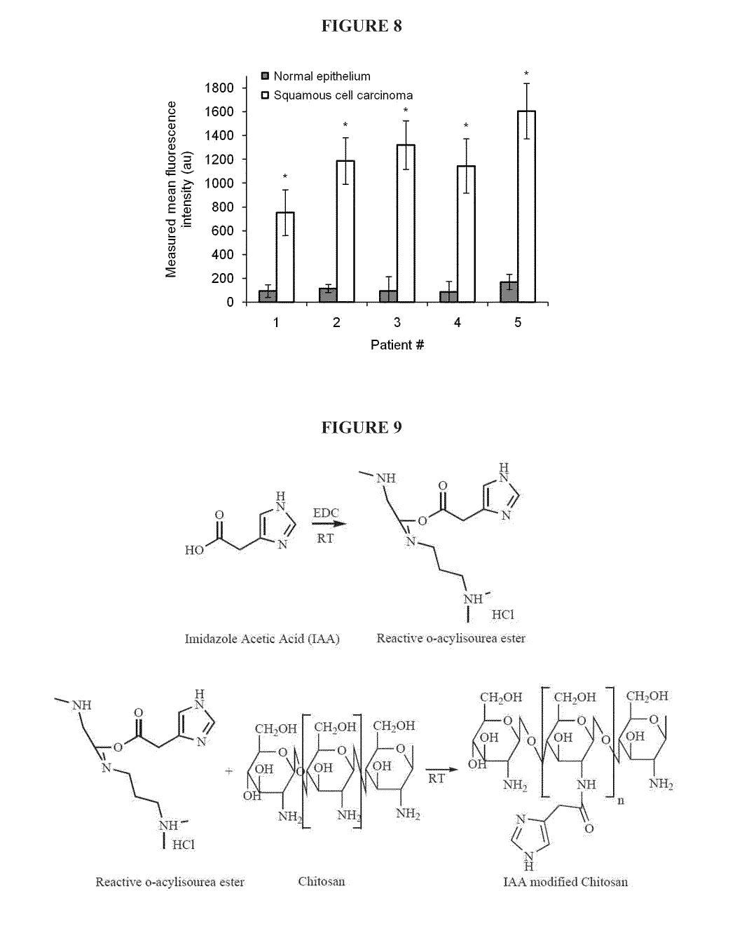

FIG. 8 shows the mean fluorescence intensity of paired oral biopsies collected from 5 patients. The biopsy pairs were labeled and imaged as described above. The mean fluorescence intensity of EGFR labeling in normal biopsies was generally low and showed little variation from patient-to-patient, despite 4 different anatomic sites. The intensity of EGFR labeling in squamous cell carcinoma biopsies showed a statistically significant (p≦0.01) difference when compared to their respective contralateral controls. The relative contrast ratio of cancer-to-normal tissue was found to be 8.1, 10.4, 14.0, 13.3, and 9.5 for patients 1 to 5 respectively.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A delivery formulation comprising:
a chitosan in which the primary amines are partially modified with 4-imidazole acetic acid monohydrochloride; and
a non-nucleic acid therapeutic or imaging material.

2. The delivery formulation of claim 1, wherein non-nucleic acid therapeutic or imaging material comprises an antibody.

3. The delivery formulation of claim 1, wherein the non-nucleic acid therapeutic or imaging material comprises an optical imaging agent.

4. The delivery formulation of claim 1, wherein the non-nucleic acid therapeutic or imaging material comprises a metal nanoparticle.

5. The delivery formulation of claim 1, wherein the chitosan is bound to the non-nucleic acid therapeutic or imaging material.

6. The delivery formulation of claim 1, wherein the chitosan is not bound to the non-nucleic acid therapeutic or imaging material.

7. A method for delivering a material to an epithelial or mucosal tissue comprising increasing the permeability of an epithelial or mucosal tissue by applying to said tissue a delivery formulation comprising:
a chitosan in which the primary amines are partially modified with 4-imidazole acetic acid monochlorohydride; and
a non-nucleic acid therapeutic or imaging material.

8. The method of claim 7, wherein applying comprises applying the delivery formulation to said tissue in vivo.

9. The method of claim 7, wherein applying comprises applying the delivery formulation to said tissue in situ.

10. The method of claim 7, wherein the mucosal or epithelial tissue comprises oral mucosa.

11. The method of claim 7, wherein the mucosal or epithelial tissue comprises bladder tissue.

12. The method of claim 7, wherein the material comprises an imaging material and the method further comprises detecting the imaging material in said tissue.

13. The method of claim 12, wherein the imaging material comprises an optical imaging material and detecting comprises confocal microscopy.

14. The method of claim 7, further comprising rinsing said tissue to remove the delivery formulation.

15. The method of claim 7, wherein the non-nucleic acid therapeutic or imaging material comprises an antibody.

16. The method of claim 7, wherein the non-nucleic acid therapeutic or imaging material comprises a metal nanoparticle.

17. The method of claim 7, wherein the chitosan is bound to the non-nucleic acid therapeutic or imaging material.

18. The method of claim 7 wherein the chitosan is not bound to the non-nucleic acid therapeutic or imaging material.
